(19) United States
(12) Patent Application Publication
(10) Pub. No.: US 2005/0208111 A1
(43) Pub. Date: Sep. 22, 2005

(54) Personal care and surface cleaning article

(76) Inventor: Albert R. Kelly, Douglaston, NY (US)

Correspondence Address: Evelyn M. Sommer;Suite 825, 250 Park Avenue, New York, NY, 10022, US

(21) Appl. No.: 11/130,565

(22) Filed: May 18, 2005

Related U.S. Application Data
(63) Continuation-in-part of application No. 10/021,395, filed on Dec. 19, 2001.

(51) Int. Cl. A61K 9/70; A47L 1/06

(52) U.S. Cl. 424/443; 15/209.1

(57) ABSTRACT

A flexible, substantially dry, disposable article suitable as a personal care article for cleansing and conditioning skin, sanitizing and disinfecting hard surfaces, methods of manufacturing and using the same wherein the article comprises: first, a water insoluble flexible nonwoven thermoplastic outer layer having an outer and inner surface; a core layer possessing low density, high absorbency, and high stretch capacity, containing a treatment composition adapted for cleaning, sanitizing or disinfecting hard surfaces or for personal cleansing and conditioning and a second water insoluble flexible nonwoven thermoplastic layer having an outer and inner surface positioned so its inner surface is opposite the inner surface of the first layer; the core layer having been ultrasonically through-bonded to and between the first and second layers so as to form a unified article having pin dot perforations of an average pore opening size of less than about 300 microns and preferably less than 100 microns in diameter formed by the ultrasonic bonding extending through all three of said layers. The invention also contemplates the articles which have not been treated with a treatment composition.

This application is a continuation-in-part of application Ser. No. 10/021,395, filed Dec. 19, 2001.

### FIELD OF THE INVENTION

The present invention relates to substantially dry, flexible disposable articles suitable as personal care articles for cleaning and conditioning skin or for cleaning, sanitizing and disinfecting hard surfaces, methods for manufacturing and for using the same.

This invention more particularly relates to a disposable article useful for personal care and cleaning hard surfaces comprising a first water insoluble flexible nonwoven thermoplastic layer, a second water insoluble flexible nonwoven thermoplastic layer, an intermediate core layer of a cellulosic wadding or tissue material, the core layer being ultrasonically bonded to and between the first and second layers so as to form a unified article having pin dot perforations extending through all three layers.

The invention is also directed to such substantially dry flexible disposable articles having a cleaning or conditioning solution incorporated in the core layer.

Treated flexible articles for personal care and for cleaning, sanitizing and disinfecting hard surfaces are known. These articles are generally single ply structures that have been coated, sprayed, or impregnated with a treatment composition that is activated on being wetted with water. The articles with the treatment composition incorporated therein are substantially dry to the touch. A disadvantage of these articles is that when large surfaces are involved or when it is desired that the article be used for more than one use, necessitating the rewetting of the article to reactivate the treatment composition incorporated therein, that on the first wetting of the article, the active ingredients are substantially completely released such that the intended larger or multi-stage cleaning task cannot be completed. Another disadvantage of the available cleaning articles is that the use of higher levels of treatment composition adversely affects hand-feel, the heavily treated articles feeling wet, lubricious or boardy.

A key requisite for producing longer-lasting cleaning articles is inclusion of enough treatment composition for the tasks intended, and provisions both for accelerating activation of the treatment composition by water and for controlling the release of the activated treatment composition over time. Traditionally, this had been accomplished with the use of concentrated treatment compositions that contain little or no water, that are solid or semi-solid at room temperatures, that are coated onto the surface of a flexible substrate at elevated temperatures, and then allowed to dry as a thin coating at ambient temperatures. The disadvantages of such coatings include lubricity of the resultant wax-like coatings, boardy hand-feel and an inability to control activation or release of the treatment composition; all of which depend upon the nature of the substrate and of the chemistry of the treatment compositions.

The bonding of nonwoven layers to form a unified article can be achieved in three major manners: chemical bonding, thermal bonding and mechanical bonding. The recent development in this field has seen growing shares of thermal bonding. Typical methods of thermal bonding include hot calendering, belt calendering, oven bonding, ultrasonic bonding, radiant-heat bonding and etc. Thermal bonding processes, including ultrasonic bonding processes, enjoy a range of advantages over other bonding processes, such as producing a soft and textile-like product, high economic efficiency as compared to chemical bonding with binder agents because no water evaporation is required, less expensive machinery in terms of lower capital expenditure, maintenance and operating costs, the possibility of bonding thicker webs uniformly and thoroughly to the core which cannot be achieved mechnically, the absence of necessity for binder agents or curing processes (no exhaust air or waste water), the possibility of using pure polymer fibers or blends so that a recyclability of practically 100% is obtainable, and etc.

Ultrasonics is the science of the effects of sound vibrations beyond the limit of audible frequencies. The object of high power ultrasonic applications is to bring about some permanent physical change in the material treated. This process requires the flow of vibratory energy per unit of area or volume. Depending on the application, the resulting power density may range from less than a watt to thousands of watts per square centimeter. Although the original ultrasonic power devices operated at radio frequencies, today most operate 20,000-69,000 Hz.

Ultrasonics is used in a wide variety of applications. For example, ultrasonics can be used for dust, smoke and mist precipitation; preparation of colloidal dispersions; cleaning of metal parts and fabrics; thermoplastic bonding; the formation of catalysts; the degassing and solidification of molten metals; metal welding; the extraction of flavor oils in brewing; electroplating; drilling hard materials; fluxless soldering; and nondestructive testing such as in diagnostic medicine.

Ultrasonic bonding of fibrous materials is known and comprises a process which involves the application of rapidly alternating compressive forces to localized areas of fibers in the web. The stress created by these compressive forces is converted to thermal energy, which softens the fibers as they are pressed against each other. Upon removal from the source of ultrasonic vibration, the softened fibers cool, solidifying the bond points. This method is frequently used for spot or patterned bonding of mechanically bonded materials. No chemical binder is necessary when synthetic fibers are used since these are self-bonding. To bond natural fibers, some amount of synthetic fiber must be blended with the natural fiber. Fabrics produced by this technique are soft, breathable, absorbent and strong. This bonding method is used to make patterned composites and laminates.

The use of ultrasonic forces in welding sheet materials have been known in the art as for example in the patent literature.

U.S. Pat. No. 3,660,186 teaches a continuous method for bonding or seaming running webs.

U.S. Pat. No. 3,697,357 teaches welding sheets made entirely or partially of thermoplastic material or fiber by sealing an area of material.

U.S. Pat. No. 3,939,033 teaches using ultrasonics to simultaneously seal and cut thermoplastic textile material.

U.S. Pat. No. 5,061,331 discloses an ultrasonic cutting and edge sealing apparatus for cutting and sealing semi-permeable and at least partially thermoplastic fabrics.

Ultrasonic force has also been used to perforate sheet materials. U.S. Pat. No. 3,966,519 teaches perforating non-woven webs. The ultrasonic energy amount is controlled by applying a fluid to the area where the ultrasonic energy is applied.

U.S. Pat. No. 3,949,127 teaches perforating non-woven webs by applying intermittent ultrasonic fusion to the web and then stretching the web to break the most intensely fused regions causing perforations to form in the web.

U.S. Pat. No. 5,269,981 teaches microaperturing a thin sheet material which requires applying a liquid to the thin film before subjecting it to ultrasonic vibrations.

U.S. Pat. No. 5,735,984 teaches forming apertures, about 200 microns in diameter, in an adhesive coated non-woven or foam sheet where the height of the flattened raised areas on the anvil is equal to or less than the thickness of the sheet material and adhesive.

Aperturing thin sheets using ultrasonics is desirable because it allows rapid movement of the thin sheet through the process without creating waste. In the past, methods used to aperture thin sheets have included punching the sheet material. Although punching the aperture out of the sheet material created the desired perforated effect, punching left behind residual waste in the form of cores. The cores often adhered to the thin sheet material and produced an undesirable effect because they interfered with manufacturing of the final product.

An object of this invention is to provide a substantially dry, flexible cleaning article useful for cleaning, sanitizing and disinfecting hard surfaces that can be activated with water and reactivated for continued use or repeated use, until the cleaning task or tasks are completed.

Another object of this invention is a flexible, substantially dry, cleaning article having incorporated into its structure, treatment composition in an amount of from 25 to 300 percent of the structure's total basis weight without the hand feel being adversely affected and without any loss of treatment composition from the article prior to actual use thereof.

It is another object of the invention to provide such cleaning articles which are disposable without giving rise to ecological or other problems.

Yet another object is to provide a cleaning article adapted for controlled and repeated release of the active ingredients present in the treatment composition incorporated in the articles' structure.

Still another object is to provide a cleaning article adapted for controlled and repeated release of the active ingredients present in the treatment composition incorporated in its structure and adapted for use for personal care.

Yet another object is to provide a cleaning article adapted for controlled and repeated release of the active ingredients present in the treatment composition incorporated in its structure and adapted for use for cleaning, sanitizing, and disinfecting hard surfaces.

Still a further object of the invention is to provide a cleaning article adapted for controlled and repeated release of the active ingredients present in the treatment composition incorporated in its structure having different surface textures on opposite sides thereof

Yet another object of the invention is to provide a cleaning article adapted for controlled and repeated release of the active ingredients present in the treatment composition incorporated in its structure having an outer surface that can be imprinted for labeling, decorative or instructional purposes.

Disposable articles as herein disclosed are intended for single use, as well as multiple uses, i.e., up to 12 and more uses and as such are more disposable than reusable sponges, cloths or pads which can develop bacterial growth, unpleasant odors and other undesirable characteristics.

These and other objects will become readily apparent from a reading of the detailed description of the invention that follows.

### DETAILED DESCRIPTION OF THE INVENTION

A disposable flexible article useful for cleaning, sanitizing and disinfecting hard surfaces or for personal cleansing and conditioning, adapted for inclusion of a cleaning composition or cleansing or conditioning composition, respectively in the core layer thereof and for controlled and repeated release thereof, comprising:
a) a first water insoluble nonwoven layer comprised substantially of synthetic fibers having outer and inner surfaces,
b) a water insoluble core layer made of cellulosic material having a density less than 0.7, preferably less than 0.3 and most preferably less than 0.2 grams per cubic centimeter, having a stretch capacity of at least 10%, preferably up to 75%, having a water absorbent capacity of at least 6, preferably up to 17 and most preferably 12-15 grams per gram of its basis weight, and
c) a second water insoluble nonwoven layer comprised substantially of synthetic fibers having outer and inner surfaces
d) wherein the core layer is positioned between the inner surfaces of said first and second layers and the first, second and intermediate core layer are ultrasonically bonded to form a unified article having perforations of less than 300 microns in diameter extending through all of said layers, said perforations having been formed during said ultrasonic bonding, wherein said perforations assist in controlling water absorption into the core required for activating the cleaning, cleansing or conditioning composition and for the controlled and repeated release of the cleaning, cleansing or conditioning composition upon its activation by said absorbed water.

The invention also contemplates flexible substantially dry disposable articles comprising a first water insoluble flexible nonwoven thermoplastic outer layer having an outer and an inner surface, a core layer formed at least in part of a cellulosic material possessing low density of less than 0.7, preferably less than 0.3 grams per cubic centimeter and most preferably less than 0.2 grams per cubic centimeter, high absorbent capacity and high stretch capacity, containing a treatment composition adapted for cleaning, sanitizing or disinfecting hard surfaces or for personal cleansing and conditioning and a second water insoluble flexible nonwoven thermoplastic layer having an outer and inner surface positioned so its inner surface is opposite the inner surface of the first layer, the core layer being ultrasonically through-bonded to and between the first and second layers so as to form a unified article having perforations at less than 300 microns in diameter formed by said ultrasonic bonding.

When the term “thermoplastic layers” is used, it is intended to include layers, which are entirely or substantially composed of thermoplastic fibers. It is however possible for the thermoplastic layer to include lesser amounts of natural fibers such as rayon, cotton, viscose, lyocell or Tencel®.

Cellulosic materials suitable for use in forming the core layer are fibers composed or derived from cellulose. Examples are paper (cellulose), hemp and cotton. Particularly preferred fibers are derived from wood pulp. The cellulosic core can be a wadding, a tissue, or other like cellulosic materials. It is critical for the repeated use of the wipe to employ a lightweight and highly absorbent core structure. The desired cellulosic material, a wadding or a tissue, possesses a density of less than 0.7, preferably less than 0.3 grams per cubic centimeter and most preferably no more than 0.2 grams per cubic centimeter and can absorb up to 17 grams per gram of its basis weight, preferably 12 to 15 grams per gram of its basis weight. Conventional papers have densities well in excess of 0.3 grams per cubic centimeter, and an absorbency of up to about 7 grams per gram of their basis weight. Because it is essential for the core to retain a relatively large amount of treatment composition, generally more than 6 grams per wipe, to allow for repeated use, the use of a low absorbency cores yield wipes which become nearly completely or over saturated, when the required amount of treatment approaches or exceeds the absorption capacity of the core. Thus, conventional paper cores, with an absorbent capacity of up to 7 grams per gram of its basis weight, will yield a wiper product that can retain, but will more quickly release the 6 grams of composition needed to be effective. This can give rise to more rapid activation and release of the treatment composition and a reduced span of use.

The preferred core is a cellulosic wadding core or a tissue. The preferred wadding material possesses a basis weight of 22 lbs/ream to 35 lbs/ream, preferably 28 lbs/ream, that has an unusually high stretch capabilities of about 75%, and a low density of less than 0.2 g/cm³, preferably less than 0.1 g/cm³. Shawano Specialty Papers produces cellulosic wadding of this type as Grade 1804. These characteristics allow the core to survive ultrasonic bonding and preserve structural integrity of the composite structure under the multidirectional tensions during converting and in-use. The high absorption capacity of up to 17 grams of water per gram of basis weight, or 27.2 grams of water per 7″×8″ wipe, is also critical in allowing the core to absorb and retain 6 grams of cleaning composition (representing about 22% of its capacity) without risking treatment composition migration, or its premature release.

A preferred tissue material is obtained from Cellutissue of 10.5 grams/sq. yd.

While ultrasonic bonding has been available for some time, the technique has been applied to entirely or partially thermoplastic material or fiber. Ultrasonic bonding with cellulosic material, and particularly wood pulp derived materials, was not believed possible without encountering considerable difficulties. The inventors herein have now found that the composite of the invention, with inner and outer nonwoven thermoplastics enclosing therein a low density wadding or tissue core with high stretch and high absorbency capacity, can be ultrasonically bonded together without compromising the structural integrity of the cellulosic core during ultrasonic application or when subjected to tensions under high speed converting or in-use. At the same time, discrete selection of anvils and sonic horns can result in the creation of perforations with the desired diameters for controlled absorption of water for activation of the treatment composition, and the controlled release of the activated treatment composition over time. One object of the ultrasonic bonding is to join together the different fiber webs or layers by creating bonding spots where the fibers are pressed and fused, the nature of the cellulosic web having been predetermined to insure that it will maintain its integrity during the ultrasonic process. The other object of the ultrasonic bonding is to provide perforations of desired sizes and density to allow the metered absorption of water and the controlled release of the activated treatment composition over time, provided that anvils and sonic horns of specific sizes and arrangements are employed.

In another aspect of the invention, additional layers of thermoplastic materials such as dry laid resin bonded fabrics, heat bonded fabrics, spun bonded or hydro-entangled fabrics composed of polypropylene and/or polyester, or needle punched polypropylene and/or polyester may be disposed adjacent to the outer layers or used to form the bottom surface or the top surface, of the cleansing article respectively so that articles of 4, 5, 6 or seven layers are possible, however in all instances the innermost three layers are those described herein.

Preferably, the first and second outer layers are composed essentially of thermoplastic fiber based nonwovens selected from polyesters, polyolefins, polyamides and polypropylene and most preferably spun laced, hydro-entangled polyester, or needlepunched polyester or polypropylene. The fibers can be made of single polymer component or can be bio-component fibers having a side-by-side or sheath core structure. The first and second outer layers or additional layers provided on the first and second layers can also be comprised of a mixture of the thermoplastic fibers having abrasive particles incorporated therein or which can be flame treated or singed to form roughened or hardened surfaces. The abrasive particles are added in order to form a rough or abrasive surface on at least one face of the article. Abrasive particles can be created by melting locally thermoplastic polymers thus creating polymer hard points through partial fusion of fiber ends or fiber bundles. The heat treatment applied can be a locally intense heating provided by an open flame in a process called singeing or high intensity infrared heating or similar methods capable of melting only parts of the fibrous structure. Another aspect of this invention is to apply resin-finishing treatments on the outer surface of the desired layer taking care of hardening only locally that layer.

In accordance with another embodiment of this invention a fabric is provided containing thermoplastic fibers in order to impart roughness to the desired layer. An outer layer can also be embossed or debossed ultrasonically so that it exhibits a pattern of raised and depressed areas or surface aberrations as an alternative to including abrasive particles or flame treating and the like. This abrasive or embossed surface also serves to enhance the cleaning or exfoliating effect in the case of the personal care article and the cleaning and scouring effect in the case of the article for cleaning hard surfaces.

The thermoplastics nonwovens can incorporate coloring substances therein so that they present a varied appearance, i.e., the outer layers being of the same or different colors.

To form the disposable cleaning article of this invention, a core layer is ultrasonically bonded while it is positioned between the outer layers using conventional available ultrasonic machinery and employing conditions sufficient to achieve the bonding of the elements and to perforate all of the layers effectively. The perforations of the layers should have a diameter of less than 300 microns and preferably less than 100 microns and be rendered fluid permeable. The small perforations help control and meter water adsorption, activation, and release of the cleansing or treatment component, and thereby extend the usable life of the wipe. If large perforations were created with large outside diameters (450 to 600 microns), the usable life of the wipe would be shortened due to the faster absorption of water and the more rapid release of the activated treatment composition upon initial use. If more layers are present, the bonding and formation of apertures or perforations is achieved in the same manner and to the same effect.

The term “disposable” as used herein means that the ultrasonically through-bonded articles are designed to be re-activated a limited number of times and then discarded, preferably less than about 12 to 24 times and most preferably more than a single usage event. For the purpose of the instant invention articles, all of which are specifically designed for disposability, through-bonding enhances performance and mandates disposability by facilitating controlled de-lamination to be engineered into the product's design specifications.

The term “substantially dry”, as used herein, means that the articles exhibit a hand feel that may feel slightly lubricious, but not water-wet.

The disposable cleaning articles of the invention must comprise a water insoluble inner core element and at least two outer water insoluble surface contacting elements with at least one outer surface element on each side of the core element, wherein the article contains apertures of less than 300 microns and preferably less than 100 microns in diameter extending through all three of the elements or layers. The core element may include a cellulosic substrate or another nonwoven web, i.e., a web composed in the main of cellulosic fibers having absorbent capacity, stretch, low density, controlled release, and wet strength for use. In its preferred embodiment, the core element should have an absorbent capacity of up to 17 grams per gram of core material. The wet wicking rate of the core should be less than about 25 seconds. The basis weight of the core should be from about 20 to about 44 grams per square meter. Materials for the core are selected from the group of cellulosic, natural materials such as cotton, jute, hemp, wood pulp, and mixtures thereof, which should be of low density, less than 0.7, preferably less than 0.3 grams per cubic centimeter. It is critical for the repeated use of the wipe to employ lightweight and highly absorbent core structures. It is also critical for the core to have a high stretch capability, at least 30% and preferably up to 75%, which is necessary to preserve the structural integrity and prevent the formation of fissures in the core during ultrasonic bonding, high speed converting or in use. The desired cellulosic material preferably possess a density of less than 0.3 grams per cubic centimeter and preferably no more than 0.2 grams per cubic centimeter and can absorb up to 17 grams per gram of their basis weight, and have a stretch capability of about 75%. The core materials, be they wadding or tissue all are characterized by low density, high absorbency and high stretch capacities. By comparison, standard papers have densities well in excess of 0.3 grams per cubic centimeter, absorb only up to about 7 grams per gram of their basis weight and can stretch only by about 1. 5%. The use of conventional paper results in a wipe product that cannot retain and gradually release the high load of treatment composition required for products of this type to be effective.

The cellulosic wadding or tissue core is critically important for assuring effective performance of the treated composite for the following reasons.

1. To inhibit migration of the treatment composition incorporated therein,

2. To readily absorb and retain the treatment composition until activated for use,

3. To gradually release the treatment composition upon activation with water,

4. To expand and soften the activated wipes.

After formation of the composite article, the article can be treated in the conventional manner by impregnation, dipping, spraying or coating the article with a treatment composition, the treated article remaining substantially dry to the touch. The article may also be treated so that it includes the treatment composition comprising the active ingredients in the form of a substantially dry composition that does not permeate or impregnate either of the outer layers, and effectively resides between the inner surfaces of the first and second or outer layers.

The first and second and any additional outer layers and the core are bonded together to maintain the integrity of the article and to provide the article in the thermoplastic outer layers with apertures such that the composite layers become fluid permeable. The apertures encourage the introduction of water or other fluids into the core to activate the treatment composition that has been impregnated, coated, sprayed or otherwise incorporated therein. The application of water, pressure and friction facilitates and controls the metered release of the activated composition.

The bonding of the core with the outer layers is executed thermally by ultrasonic bonding and has to be conducted so all of the layers are completely through-bonded and through-perforated. Ultrasonic bonding is conducted in the conventional manner, utilizing the conventional ultrasonic bonding equipment and anvils and horns of appropriate sizes and shapes adapted for producing the through perforations of desirable sizes as described herein.

The hard surface cleaning and cleansing/moisturizing articles of the invention contain in the core layer a treatment composition which has been added onto or impregnated and penetrates into the core layer and is releasably associated therewith.

The treatment composition for the article to be used for personal cleansing can include, in addition to surfactants and preferably lathering surfactants, emollients, lubricants, protectants, deodorants or medicaments.

The apertures should preferably be uniform in size and shape and necessarily of small size, preferably having an average diameter of less than 300 microns and most preferably less than 100 microns. In addition to the two layers and core, other insoluble layers may be present disposed adjacent to the outer surface(s) of the two layers. These additional layers can include abrasives and other non-fibrous materials. The bonding of the multi layers takes place ultrasonically and is regulated so as to bond the layers and to produce apertures or openings extended into and through all of the layers.

The articles intended for cleaning hard surfaces can have incorporated herein compositions as described in U.S. Pat. No. 6,141,644 the entirety of which is incorporated herein by reference.

The treatment composition for the article when it is to be used for cleaning, sanitizing and disinfecting hard surfaces, can include, in addition to surfactants, an antiseptic, antibacterial, wax, waterproofing, polishing or other agents as are conventionally used in cleaning compositions intended for cleaning and protecting hard surfaces in homes, restaurants, hospitals, nursing homes for private and industrial use and the like. They can be used as mopping and dusting cloths, and as polishing and cleaning cloths.

In a preferred embodiment for personal skin care, the treatment composition includes a surfactant and at least one member of the group of emollients, lubricants, conditioning agents protectants, deodorants and medicaments. When the article is exposed to water at the point of use and pressure applied, as by squeezing, an unstable emulsion and in the case of the use of a foaming surfactant, a lather is formed which releases the components of the cleansing treatment composition onto the skin or hair of the individual. The treatment compositions used in connection with personal skin care articles is most advantageously used to provide 25 to about 300 percent of add-on treatment composition based upon total basis weight of the final product.

The treatment composition for cleaning, sanitizing and disinfecting hard surfaces and for cleansing and conditioning the skin can further include a visual indicator, such as a water soluble colorant or dye, whose disappearance signifies the depletion of the treatment composition upon repeated application. For example, sodium thiosulfate and various blue dye mechanisms, such as those employed in the WIPEX® wipes may be employed. The water soluble colorant or dye can be independently dissolved or dispersed in the treatment composition and is released gradually during use and depleted at about the same time the active ingredients in the treatment composition are exhausted. The colorant or dye can also bond to one or more of the active ingredients at a molecular scale so that the release of dye is attendant to the release of the active ingredients. The molecular bonding between the colorant or dye with the active ingredients offer a more accurate indication and such examples can be found in U.S. Pat. No. 4,311,479. The indicator system can also include at least one dye component and a polymer mixture as disclosed in U.S. Pat. appl. Ser. No. 2001/0031595. It is also conceived that the colorant or dye can be covalently or noncovalently attached to one or more layer of substrate and the depletion of the treatment composition can be indicated by the change in the color of the colorant or dye as a result of the change in the environment, such as a change in the pH value of the medium as the treatment composition is diluted and depleted. The indicator systems can also include time indicators, chemical indicators, dye solubility indicators and binder/dye indicators as disclosed in U.S. Pat. No. 6,734,157. The indicators can be present throughout the treatment composition or on one or more layers of the substrate, or can be present in various patterns, such as stripes.

The outer layers of the article of the invention may have different textures and abrasiveness. Differently textured surfaces can be adapted so as to provide an abrasive side for more intensive cleaning and a softer absorbent side for lighter and more gentle cleaning.

The surfactants employed in the treatment compositions are preferably lathering surfactants, but this is not required and may include anionic, cationic, amphoteric and non-ionic surfactants either separately or in combination and preferably include as anionic surfactants, the phosphates, taurates, sulfates, sarcosinates, isothionates, etc, for example ammonium lauroyl sulfate, sodium lauroyl sarcosinate, sodium lauroyl lactylate, etc.

Nonionic surfactants which can be used include amine oxides, alkoxylated fatty acid esters, polyhydroxy fatty acid amides, alkyl glycosides and the like exemplified by lauramine oxide, sucrose laurate, sucrose cocoate and the like.

Instances of suitable amphoteric surfactants are the amino alkanoates, alkyl amino acetates, hydroxy, sultaines, betaines, etc., as exemplified by sodium-3-dodecylaminopropinate, disodium lauroamphodiactetate and sodium lauroampho acetate.

The substantially dry core is interposed between the two water insoluble outer layers, and the resultant lamination is ultrasonically bonded to form a perforated uniform article and is then treated with the treatment composition.

Preferably the article of the invention comprises three discrete elements that have been ultrasonically through-bonded to form a uniform perforated article. For the purpose of the ultrasonical bonding, the outer layers are preferably composed of similar synthetic fiber materials and the inner layer or core layer of a cellulose material and preferably a wood pulp derived material such as paper.

The disposable articles of the invention can accommodate from 25 to about 300 percent of treatment composition of the resulting structures' total basis weight without any loss of their excellent hand properties including feel, drape conformability and flexibility.

The disposable articles of the invention are substantially dry and are intended to be activated by water prior to use by saturation with water, by placing the article under a stream of water or by placing it in contact with a wet hard surface or wet skin. Upon activation with water the treatment composition is released by applying pressure and/or friction.

The articles all have in common a high absorption capacity facilitated by use of the absorbent cellulose fiber core. The apertures formed in the ultrasonic bonding encourage rapid passage of water into and for gradual release of the treatment composition out of the device, the apertures being capable of modification to influence how the water gets in the article and amounts of the composition out of the article.

In order to describe the invention more fully and not by way of limitation, the following examples are presented.

1. Three-Ply Hard Surface Cleaning Cloths

A single inner absorbent core element comprising a 28# cellulosic wadding, having a density of about 0.1 gram per cubic centimeter, that is interposed between a web of 1.2 ounce per square yard polyester material made of 3 denier fibers and 2.3 ounce polypropylene fiber material made of a blend of coarse fibers of 17 and 60 denier. These three elements are ultrasonically bonded together to form a flexible article of controlled density. A calendar compression treatment is applied between two metallic rolls in order to achieve a thickness of 1/20^{th} of an inch and a pore opening of less than 100 microns. The resulting cloth when wet with water, and wrung dry provided an excellent cleaning device. The cloth exhibited a high water absorption capacity. It could be wet and wrung out many times without depleting its absorbency being reduced.

2. Three-Ply Hard Surface Cleaning Cloths

A single inner absorbent core element comprising a 28# cellulosic wadding material, having a density of about 0.1 gram per cubic centimeter, that is interposed between a web of 1.2 ounce per square yard of polyester material made of 3 denier fibers and a 2.3 ounce polypropylene fiber material made of blend of coarse fibers of 17 and 60 denier. The three elements are ultrasonically bonded together to form a flexible article of controlled density. A calendar compression treatment is applied between two metallic rolls in order to achieve a thickness of 1/20^{th} of an inch and a pore opening size of less than 100 microns.

Because the treatment composition is impregnated under pressure, and the treated composite is passed through a series of nip rolls and folding ploughs, over 80% of the treatment composition is absorbed into the cellulosic core. This treated core element weighs approximately 174 grams per square yard. The resulting cloth is then placed under running water and squeezed several times to provide a rich lathering foam providing excellent cleaning properties. The process was repeated approximately twelve times and still provided a rich lathering foam. This process was repeated approximately twelve times and still provided a rich lathering foam. This process was repeated twelve more times, at which point the lathering foam diminished.

3. Four-Ply Hard Surface Cleaning Cloths

A single, inner absorbent core element comprising a 28# cellulosic wadding, having a density of about 0.1 gram per cubic centimeter is imposed between a 1.2 ounce per square yard carded and cross-lapped polyester fabric made of 3 denier fibers and a 2.3 ounce polypropylene fiber material made of 17 denier fibers. The three elements are ultrasonically bonded together to form a flexible article of controlled density. A calendar compression treatment is applied to the laminate between two metallic rolls in order to achieve a thickness of 1/20^{th} of an inch and an average pore opening size of less than 100 microns. The compressed fabric is subjected to an open flame treatment in order to impart the abrasion properties to the composite material. The article was impregnated with 90 grams per square yard of a highly concentrated hard surface surfactant containing treatment composition. This treated core element weighs approximately 126 grams per square yard and contains the activated treatment composition for ready release. The treated cloth is then saturated by immersion in water and squeezed several times to produce a rich lathering foam. This process was repeated over 24 times before the foaming action began to dissipate.

4. Four-Ply Hard Surface Cleaning Cloths

A single, inner absorbent core element comprising a 28# cellulosic wadding material, having a density of about 0.1 gram per cubic centimeter is interposed between a 1.3 ounce polypropylene material which has been interfaced with a 0.5 milligram polyethylene film and a 1.0 ounce spun-bonded polyester material. The four layers are then bonded together ultrasonically so as to form a flexible entity having perforated bonding points of less than 200 microns in diameter arranged in uniform pin-dot pattern through which fluids can pass into the treated core and the activated treatment following its delivery can be released outwardly. The perforated polyethylene film layer is intended to present a partial fluid barrier that restricts in use the inward flow of water and the subsequent release of activated treatment composition. The article is coated in three-quarter inch wide stripes with 60 grams per square yard of a highly concentrated hard surface surfactant containing composition, separated by one-half inch stripes having no coating. The treated article was then saturated with water and squeezed several times to produce a rich lathering foam. This process was repeated over 24 times before the foaming action began to dissipate.

5. Three-Ply Skn Cleansing Cloths

A single, inner absorbent core element comprising a 35# cellulosic wadding material weighing approximately 48 grams per square yard is interposed between a 1.3 ounce resin-bonded, polyester material and a 1.0 ounce spun-bonded polyester material. The three elements are then ultrasonically bonded together so as to form a flexible entity having perforation points with a diameter of less than 150 microns arranged in uniform pin-dot pattern that is visible on the spun-bonded polyester side only. The cloth is impregnated with 140 grams per square yard of a concentrated skin cleansing and conditioning composition. The treated article is then saturated under running water and squeezed several times to provide a rich, lathering foam. This process was repeated approximately 42 times, at which point the lathering foam diminished significantly.

6. Five Ply Body Cleansing and Skin Moisturizing Cloth

A 3-ply, inner absorbent core material, comprised of 2 outer plies of 0.4 oz./sq. yd. tissue from Cellutissue with one inner ply of 0.5 oz./sq. yd. thermal bond polypropylene from Tenotex, is ultrasonically sealed between two outer plies of 0.5 oz./sq. yd. polypropylene material to form a flexible 2.3 oz. composite, having perforations of less than 150 microns arranged in a pin-dot pattern. Towels measuring 7″×8″ are impregnated with up to 6 grams of the cleansing and moisturizing concentrate. The disposable cloth was then saturated, squeezed and rubbed over the skin and used like a personal washcloth to effectively clean and moisturize the entire body, leaving the skin feeling clean and soft.

7. A Four-Ply, Hard Surface, Cleaning and Sanitizing Wipe

A two-ply, inner absorbent core with a 0.75 mil barrier film coating on the wire side facing outwardly, comprising #28 cellulosic wadding material, with a total basis weight of 56 grams/sq. yd., was impregnated with 10 grams/sq. yd. of a non-aqueous hard surface sanitizing concentrate containing a green dye, prior to being ultrasonically laminated between two plies of 17 grams/sq. yd. thermally bonded polypropylene material. The four plies were through bonded with perforations at the bonding points with pore opening of less than 200 microns in diameter, through which restricted amounts of water enter the core to activate the impregnated treatment solution, and through which limited amounts of the activated cleaning and sanitizing treatment solution are dispensed to the treated hard surface.

An 8″×12″ wiper prepared in this manner, weighing about 5 grams and containing approximately 1 gram of a quaternary (“quat”) based sanitizing solution within the interior core, was observed to absorb about 10 grams of water. The wetted wiper was then squeezed several times to express the activated exudate which was then titrated for parts-per-million quat. After being activated with water, expressed and reactivated 12 times, the parts-per-million quat titrated with the exudate from each expression activated averaged in excess of the 600 parts-per-million quat which is normally needed to effectively sanitize hard surfaces.

The following experiments were also conducted to measure the water absorbency of the wipes, to determine the capacity of the wipes for holding the actives and to evaluate the rate at which the actives are released from the wipes during use.

Wipes prepared using two different cores, 10.5 grams/sq. yd. of tissue from Cellutissue and 28 lb wadding from Little Rapids, were compared.

A trilaminate fabric with wadding as the core was prepared using 1 oz. sq. yd. Spunbonded polypropylene from Avgol and 15 grams/sq. yd. thermally bonded polypropylene from Tenotex as the thermoplastic layers and the 28 lb. wadding from Little Rapids as the core. The three layers account for 37.51, 19.88 and 42.61% of the weight of the trilaminate fabric, respectively. The measured basis weight of the laminate is 75.45 grams/sq. yd. The fabric was cut into wipes measured 8×7 inches and the wipes had an average weight of about 3.26 grams.

A five-ply laminated fabric with tissue in core was prepared using 3 plies of 15 grams/sq. yd. thermally bonded polypropylene from Tenotex and 2 plies of 10.5 grams/sq. yd. of tissue from Cellutissue (as in example #6). The polypropylene and tissue plies each account for 68.18 and 31.82% of the weight of the laminated fabric. The measured basis weight of the laminate is 76.37 grams/sq. yd. The fabric was cut into wipes measured 8×7 inches and the wipes had an average weight of about 3.3 grams.

Water absorbency of the wipes were measured by placing the wipe in a pail of water for 1 minute, removing the wipe, letting it drip and then weighing the wipe. The trilaminate wipe with the wadding core showed an absorbency of 6.58 grams of water per gram of wipe and 14.02 grams of water per gram of wadding. The 5-ply wipe with the tissue core showed an absorbency of 7.08 grams of water per gram of wipe and 22.25 grams of water per gram of tissue.

To determine the wipes' capacity in holding the actives, three different active compositions were employed (Plantapon® 611-L, Dehypound® W-82, and Emulgade®, all of Cognis).

TABLE 1
Evaluation of the Wipes' Capacity for Holding the Actives

| | Wipes | |
|---|---|---|
| | with wadding core | Wipes with tissue core |
| Add-on of Plantapon 611-L | 6.05 | 6.05 |
| (grams) | | |
| Add-on of Dehypound W- | 6.10 | 6.10 |
| 82 (grams) | | |
| Add-on of Emulgade | 6.04 | 6.04 |

For the evaluation of the rate at which the actives are released from the said trilaminate and 5-ply wipes during use, two different active compositions (Plantapon® 611-L and Emulgade®, both of Cognis) were employed. The wipes were saturated under a running faucet, and then squeezed into a pail to collect the fluid. Wipes were then quickly resaturated under running faucet and exudates squeezed into pail. The total weight of exudates was measured and the number of squeezes to transfer active out of the wipes was recorded. Depleted wipes were placed in pail of water to saturate the wipes and then removed from the water and allowed to drip off excess. Saturated weight was recorded. The results are listed in Tables 2 to 5.

TABLE 2
Evaluation of Release of Plantapon 611-L from Wipes

| | Wipe | Wipe |
|---|---|---|
| | with tissue | with wadding |
| | core | core |
| Weight of wipe (grams) | 8.6 | 8.4 |
| Grams of water to wet wipe | 10.2 | 10.3 |
| Number of squeezes to exhaustion of active | 25 | 30 |
| Total weight of water collected (grams) | 188 | 220 |
| Saturated weight of wipe (grams) | 20.2 | 20.2 |

TABLE 3
Repeat Evaluation of Release of Plantapon 611-L from Wipes

| | Wipe | |
|---|---|---|
| | with tissue | Wipe with wadding |
| | core | core |
| Weight of wipe (grams) | 8.9 | 9.3 |
| Grams of water to wet wipe | 11.7 | 13 |
| Number of squeezes to deplete active | 31 | 34 |
| Total weight of water collected (grams) | 332 | 340 |
| Saturated weight of wipe (grams) | 19.6 | 19.3 |

TABLE 4
Evaluation of Release of Emulgade from Wipes

| | Wipe | |
|---|---|---|
| | with tissue | Wipe with wadding |
| | core | core |
| Weight of wipe (grams) | 8.8 | 9.2 |
| Grams of water to wet wipe | 14 | 11.5 |
| Number of squeezes to deplete active | 8 | 9 |
| Total weight of water collected (grams) | 81 | 90 |
| Saturated weight of wipe (grams) | 22.2 | 19.9 |

TABLE 5
Repeat Evaluation of Release of Emulgade from Wipes

| | Wipe | |
|---|---|---|
| | with tissue | Wipe with wadding |
| | core | core |
| Weight of wipe (grams) | 9 | 9.2 |
| Grams of water to wet wipe | 11.3 | 14 |
| Number of squeezes to deplete active | 8 | 9 |
| Total weight of water collected (grams) | 76 | 92 |
| Saturated weight of wipe (grams) | 21.8 | 19.8 |

Even though the wipes prepared using wadding and tissue as core materials are functionally equivalent, wadding is more economically desirable in terms of cost of material and manufacturing procedures. The advantage of wadding arises at least partially from the fact that typically few layers of substrate are required to prepare a functionally equivalent wipe product.

1. A disposable flexible article useful for cleaning, sanitizing and disinfecting hard surfaces, adapted for inclusion of a cleaning solution in the core layer thereof and for controlled and repeated release thereof, comprising:
a) a first water insoluble nonwoven layer comprised substantially of synthetic fibers having outer and inner surfaces,
b) a water insoluble core layer made of a cellulosic material having a density less than 0.7 grams per cubic centimeter, having a stretch capacity of at least 30%, and having an absorbent capacity of at least 12 grams per gram of its basis weight, and
c) a second water insoluble nonwoven layer comprised substantially of synthetic fibers having outer and inner surfaces, wherein the core layer is positioned between the inner surfaces of said first and second layers and the first, second and intermediate core layers are ultrasonically through-bonded to form a unified article having perforations of less than 300 microns in diameter extending through all of said layers, said perforations having been formed during said ultrasonic bonding, wherein said perforations assist in controlling water absorption into the core required for activating the cleaning solution and for the controlled and repeated release of the cleaning solution on its activation by said absorbed layer.

2. A disposable flexible article according to claim 1 wherein said cellulosic material is tissue.

3. A disposable flexible article according to claim 1 wherein said cellulosic material is wadding.

4. A substantially dry disposable flexible article useful for cleaning, sanitizing and disinfecting hard surfaces, adapted for inclusion of a cleaning solution for cleaning, sanitizing and disinfecting such hard surfaces and for controlled and repeated release thereof, comprising:
a) a first water insoluble nonwoven layer comprised substantially of synthetic fibers having outer and inner surfaces,
b) a water insoluble core layer made of a cellulosic material having a density of less than 0.7 grams per cubic centimeter, having a stretch capacity of at least 30%, having an absorbent capacity of at least 12 grams per gram of its basis weight, and incorporating about 25 to about 300% of the article's total basis weight of a cleaning solution including a surfactant adapted for cleaning hard surfaces,
c) a second water insoluble nonwoven layer comprised substantially of synthetic fibers having inner and outer surfaces wherein the core layer is positioned between the inner surfaces of said first and second layers and the first, second and intermediate core layers are ultrasonically through-bonded to form a unified article having perforations of less than 300 microns in diameter extending through all of said layers, said perforations having been formed during said ultrasonic bonding, wherein said perforations assist in controlling water absorption into the core required for activating the cleaning solution and for the controlled and repeated release of the cleaning solution on its activation by said absorbed water.

5. A substantially dry disposable flexible article according to claim 4 wherein said cellulosic material is tissue.

6. A substantially dry disposable flexible article according to claim 4 wherein said cellulosic material is wadding.

7. A substantially dry disposable flexible article according to claim 4 wherein said cleaning solution contains surfactant selected from the group consisting of anionic, nonionic, amphoteric surfactants and mixtures thereof.

8. A substantially dry disposable flexible article according to claim 4 wherein said cleaning solution further contains at least one member selected from the group consisting of antiseptic, antibacterial, wax, waterproofing and polishing agents.

9. A substantially dry disposable flexible article according to claim 4 wherein at least one additional layer is present having been applied to the outer surface of at least one of said first and second layers prior to the ultrasonic bonding of the three layers.

10. A substantially dry disposable flexible article according to claim 9 wherein said additional layer serves a partial fluid barrier.

11. A substantially dry disposable flexible article according to claim 4 wherein at least one of said first and second layers contains abrasive material.

12. A substantially dry disposable flexible article according to claim 4, wherein said perforations are less than 200 microns in diameter, said perforations ensuring the controlled passage of water into the core layer for the activation of the treatment composition and the controlled release of treatment composition activated by contact with said water out of the article.

13. A substantially dry disposable flexible article useful for cleans and conditioning skin, adapted for inclusion of a cleansing and moisturizing solution for controlled and repeated release thereof, comprising:
a) a first water insoluble nonwoven layer comprised substantially of synthetic fibers having outer and inner surfaces,
b) a water insoluble core layer made of a cellulosic material having a density of less than 0.7 grams per cubic centimeter, having an stretch capacity of at least 30%, having an absorbent capacity of at least 12 grams per gram of its basis weight, and incorporating about 25 to about 300% of the article's total basis weight of a cleansing and moisturizing solution adapted for cleansing and conditioning skin,
c) a second water insoluble nonwoven layer comprised substantially of synthetic fibers having inner and outer surfaces wherein the core layer is positioned between the inner surfaces of said first and second layers and the first, second and intermediate core layers are ultrasonically through-bonded to form a unified article having perforations of less than 300 microns in diameter extending through all of said layers, said perforations having been formed during said ultrasonic bonding, wherein said perforations assist in controlling water absorption into the core required for activating the cleansing and moisturizing solution and for the controlled and repeated release thereof on its activation by said absorbed water.

14. A substantially dry disposable flexible article according to claim 13 wherein said cellulosic material is tissue.

15. A substantially dry disposable flexible article according to claim 13 wherein said cellulosic material is wadding.

16. A disposable flexible article according to claim 13 wherein said cleansing and moisturizing solution includes a surfactant selected from the group consisting of anionic, nonionic, amphoteric surfactants and mixtures thereof.

17. A disposable flexible article according to claim 13 wherein said cleaning and moisturizing solution further contains at least one member selected from the group consisting of emollients, lubricants, conditioning agents, protectants, deodorants and medicaments.

18. A method for cleaning, sanitizing and disinfecting hard surfaces using a substantially dry disposable flexible article, said disposable flexible article comprising:
a) a first water insoluble nonwoven layer comprised substantially of synthetic fibers having outer and inner surfaces,
b) a water insoluble core layer made of a cellulosic material having a density of less than 0.7 grams per cubic centimeter, having a stretch capacity of at least 30%, having an absorbent capacity of at least 12 grams per gram of its basis weight, and incorporating about 25 to about 300% of the article's total basis weight of a cleaning solution including a surfactant adapted for cleaning hard surfaces,
c) a second water insoluble nonwoven layer comprised substantially of synthetic fibers having inner and outer surfaces wherein the core layer is positioned between the inner surfaces of said first and second layers and the first, second and intermediate core layers are ultrasonically through-bonded to form a unified article having perforations of less than 300 microns in diameter extending through all of said layers, said perforations having been formed during said ultrasonic bonding, wherein said perforations assist in controlling water absorption into the core required for activating the cleaning solution and for the controlled and repeated release of the cleaning solution on its activation by said absorbed water,
consisting of the following steps:
i) wetting said disposable flexible article with water, and
ii) applying said disposable flexible article to said hard surfaces

19. A method for cleansing and conditioning skin using a substantially dry disposable flexible article, said disposable flexible article comprising:
a) a first water insoluble nonwoven layer comprised substantially of synthetic fibers having outer and inner surfaces,
b) a water insoluble core layer made of a cellulosic material having a density of less than 0.7 grams per cubic centimeter, having a stretch capacity of at least 30%, having an absorbent capacity of at least 12 grams per gram of its basis weight, and incorporating about 25 to about 300% of the article's total basis weight of a cleansing and moisturizing solution adapted for cleansing and conditioning skin,
c) a second water insoluble nonwoven layer comprised substantially of synthetic fibers having inner and outer surfaces wherein the core layer is positioned between the inner surfaces of said first and second layers and the first, second and intermediate core layers are ultrasonically through-bonded to form a unified article having perforations of less than 300 microns in diameter extending through all of said layers, said perforations having been formed during said ultrasonic bonding, wherein said perforations assist in controlling water absorption into the core required for activating the cleansing and moisturizing solution and for the controlled and repeated release thereof on its activation by said absorbed water,
consisting of the following steps:
i) wetting the skin, and
ii) applying said disposable flexible article to said wetted skin, and
iii) rinsing with skin.

20. A substantially dry disposable flexible article according to claim 4 wherein said cleaning solution further contains a visual indicator to detect the exhaustion of the cleaning solution.

21. A substantially dry disposable flexible article according to claim 13 wherein said cleansing and moisturizing solution further contains a visual indicator to detect the exhaustion of the cleansing and moisturizing solution.
